(19) United States
(12) Patent Application Publication
(10) Pub. No.: US 2025/0009925 A1
(43) Pub. Date: Jan. 9, 2025

(54) DIFFUSER DEVICE FOR DISPERSING A SUBSTANCE THAT IS IN THE LIQUID OR SOLID STATE AT ROOM TEMPERATURE INTO THE AIR AS VAPOR

(71) Applicant: CAELIMP, LABEGE (FR)

(72) Inventors: Samuel DUFOUR, TOULOUSE (FR); Arnaud JOURDAN, TOULOUSE (FR); Philippe RIVIERE, TOULOUSE (FR)

(21) Appl. No.: 18/711,061

(22) PCT Filed: Nov. 17, 2022
(86) PCT No.: PCT/EP2022/082258

§ 371 Date: May 16, 2024

(30) Foreign Application Priority Data
Nov. 17, 2021 (FR) FR2112164

(51) Int. Cl. A61L 9/03

(52) U.S. Cl. CPC A61L 9/037; A61L 9/032; A61L 2209/133; A61L 2209/135; A61L 2209/21

(57) ABSTRACT

A diffuser device disperses a substance that is in a liquid or a solid state at room temperature into the air as a vapor. The diffuser device has a receptacle that holds a storage container containing the substance. A porous body has an evaporation surface located in the receptacle. A heating member is arranged on or in the porous body so as to control a flow of the substance through the porous body. An aeration system has an air inlet, a fan and an air outlet. The aeration system is configured to create a flow of air from the air inlet to the air outlet while sweeping across the evaporation surface in an upward direction with respect to the diffuser device.

### TECHNICAL FIELD

The invention relates to the field of diffusing apparatus intended to disperse into the air in the vapor state a substance in the liquid or solid state at ambient temperature.

### PRIOR ART

A diffusing apparatus of the forementioned type is known for example from the document WO 2019/243734 A1. In that apparatus a storage container containing the substance is connected to a dispensing member. The dispensing member includes microchannels forming an outlet arranged in the channel in order to constitute therein a zone in which the substance evaporates. A heating element is arranged on or in the dispensing member in such a manner as to control a flow of the substance through the dispensing member. A fan generates a flow of air sweeping the evaporation surface. In some embodiments the dispensing member is below the storage container in the direction of the acceleration due to gravity and the flow of air generated by the fan sweeps the evaporation surface in the direction of the acceleration due to gravity.

### SUMMARY OF THE INVENTION

The invention proposes a diffusing apparatus for dispersing into the air in the vapor state a substance in the liquid or solid state at ambient temperature, the diffusing apparatus including:
a receptacle;
a storage container containing the substance and including a drain orifice, the storage container being received in the receptacle so that the drain orifice is oriented in a downward direction of the diffusing apparatus;
a dispensing member positioned at the outlet of the drain orifice and connected to the drain orifice, the dispensing member comprising a porous body having an evaporation surface and being situated beyond the drain orifice in the downward direction of the diffusing apparatus;
a heating member arranged on or in the porous body so as to control a flow of the substance through the porous body; and
an aeration system comprising an air intake, at least one fan and at least one air outlet, the aeration system being configured to create a flow of air from the air intake to said at least one air outlet and sweeping the evaporation surface in an upward direction of the diffusing apparatus opposite the downward direction.

The diffusing apparatus is intended to be used in a position of use in which the downward direction is downward and the upward direction is upward relative to the acceleration due to gravity. In the position of use the acceleration due to gravity constitutes a driving force able to contribute to generating a flow of the substance from the drain orifice to the evaporation surface, and therefore in the downward direction.

The heating member enables control of this flow of the substance through the porous body simply by heating or not heating the porous body. The physical principles behind this control of the flow of the substance through the porous body are described in the document WO 2019/243734 A1 already cited above and in the document WO 2020/254733 A1.

As described in the above documents the heat produced by the heating member reduces the dynamic viscosity of the substance, which enables the fluid to circulate in the dispensing member in accordance with the Darcy law and then to spread on the surface of said dispensing member. With no input of heat the flow is fixed because the sum of the adhesions in the dispensing member conforms to the Jurin law. In other words, flow is permitted through the dispensing member when hot but stopped at ambient temperature by the adhesion force between the fluid and the surface of the dispensing member.

To enable such flow through the dispensing member it is necessary to heat the dispensing member until a setpoint temperature is reached. The setpoint temperature may be chosen in such a manner that the substance flows at a sufficiently low flowrate to prevent the formation of droplets becoming detached from the dispensing member when the dispensing member is at the setpoint temperature (this phenomenon is referred to hereinafter as “dripping”).

However, before the dispensing member achieves thermal equilibrium at the setpoint temperature the dispensing member is in a transient regime that may last around approximately 5 minutes to 20 minutes. The applicant has found that even if the setpoint temperature is chosen to prevent dripping in thermal equilibrium dripping can nevertheless occur in the transient regime. Now, dripping is undesirable, because the substance that has dripped in this way does not evaporate as required at the level of the dispensing member, and may even be wasted if it escapes from the diffusing apparatus. This is particularly undesirable if the diffusing apparatus is used for short periods, for example of the order of one hour to a few consecutive hours, because the duration of the transient regime is then not negligible compared to the duration of use of the diffusing apparatus.

The applicant has found that if the aeration system is configured so that the flow of air sweeps the evaporation surface in the upward direction, and therefore in the direction opposite the downward direction, the flow of air tends to prevent the accumulation of liquid substance at the level of a lower part of the porous body in the downward direction. This choice of the direction of the flow of air thus makes it possible to eliminate or at least to limit dripping, even in the transient regime.

One idea behind some embodiments of the invention consists in proposing an arrangement of the dispensing member and of the storage container that facilitates placing the storage container in the diffusing apparatus.

The invention therefore also proposes diffusing apparatus for dispersing into the air in the vapor state a substance in the liquid or solid state at ambient temperature, the diffusing apparatus including:
a receptacle discharging into the open air through an opening oriented in an upward direction of the diffusing apparatus, that is to say for example oriented upward when the diffusing apparatus is in a position of use;
a storage container for containing the substance and including a drain orifice, the storage container being received in the receptacle so that the drain orifice is oriented in a downward direction of the diffusing apparatus, that is to say for example oriented downward in the position of use;
a dispensing member positioned at the outlet of the drain orifice and connected to the drain orifice, the dispensing member comprising a porous body having an evaporation surface situated in the receptacle, in particular in the position of use, and being situated beyond the drain orifice in the downward direction of the diffusing apparatus, that is to say for example oriented downward in the position of use;
a heating member arranged on or in the porous body so as to control a flow of the substance through the porous body; and
an aeration system comprising an air intake and at least one fan, the aeration system being configured to create a flow of air from the air intake to the opening and sweeping the evaporation surface.

In this diffusing apparatus the opening through which the receptacle discharges into the open air forms the air outlet of the aeration system.

In this diffusing apparatus the heating member also makes it possible to control the flow of the substance through the porous body simply by heating or not heating the porous body, as already mentioned hereinabove.

Thanks to the fact that the receptacle discharges into the open air through an opening oriented in an upward direction of the diffusing apparatus, that is to say oriented upward when the diffusing apparatus is in a position of use, it is easy to fit the storage container into the diffusing apparatus because it suffices to insert the storage container in the receptacle through the opening.

This kind of diffusing apparatus finds one particular application for diffusing a substance in a closed space such as a greenhouse or a building, for example, or in a place sheltered from precipitation. Indeed, in such places it is not a problem that the opening is oriented in an upward direction of the diffusing apparatus, that is to say for example oriented upward when the diffusing apparatus is in the position of use, because water from precipitation does not risk entering the diffusing apparatus via this opening.

Also, because the aeration system is configured to create a flow of air from the air intake to the opening and sweeping the evaporation surface, the diffusing apparatus enables good dispersion of air charged with evaporated substance into the ambient air.

Also, in this diffusing apparatus the choice of the direction of the flow of air makes it possible to eliminate or at least to limit dripping, even in the transient regime.

In accordance with some embodiments, one or the other of the diffusing apparatus described hereinabove may have one or more of the following features.

In accordance with one embodiment the receptacle includes an internal separator wall, the internal separator wall surrounding at least partially and preferably completely the storage container when the storage container is received in the receptacle and the internal separator wall being configured so that the flow of air circulates around the internal separator wall.

With this kind of internal separator wall if the evaporated substance is recondensed in the receptacle recondensation tends to occur on the internal separator wall rather than on the storage container.

In accordance with one embodiment the diffusing apparatus includes a casing, the casing includes an upper wall and a lateral wall and the opening discharges onto the upper wall.

In accordance with one embodiment the casing includes an internal wall defining the receptacle and extending from the upper wall.

In accordance with one embodiment the upper wall and the lateral wall are formed in an upper portion of the casing, the upper portion being slidably mounted so as to be able to slide relative to the lateral wall in the downward direction of the diffusing apparatus between:
a raised position in which an upper wall of the storage container is situated beyond the upper wall in the downward direction of the diffusing apparatus, that is to say for example below the upper wall, when the storage container is received in the receptacle; and
a lowered position in which the upper wall of the storage container is situated beyond the upper wall in the upward direction of the diffusing apparatus, that is to say for example above the upper wall of the storage container, when the storage container is received in the receptacle.

In accordance with one embodiment the porous body has a porosity in an interior part of the porous body lower than a porosity in an exterior part of the porous body surrounding the interior part.

In accordance with one embodiment the porous body includes a wick made of wood, textile, ceramic, polymer or a porous metal obtained by sintering a metal powder or a metal alloy powder.

In accordance with one embodiment the dispensing member is integral with the storage container.

Thus the storage container and the dispensing member may be supplied in the form of a removable assembly to be inserted in one piece into the receptacle. This makes using the diffusing apparatus even easier because it suffices to insert this removable assembly into the receptacle via the opening. However, other arrangements are possible. Thus in accordance with one embodiment the dispensing member or at least the porous body is fixed in the diffusing apparatus. In another embodiment the dispensing member can be demounted from the storage container.

In accordance with one embodiment the drain orifice is blocked by a foil and the diffusing apparatus further comprises a perforator device disposed between the porous body and the foil, the perforator device comprising a plurality of teeth and/or needles configured to perforate the foil when the porous body is moved in the direction of the foil.

In accordance with one embodiment the receptacle and the porous body are conformed so that said movement of the porous body in the direction of the foil occurs during installation of the removable assembly in the receptacle.

In accordance with one embodiment the drain orifice is at a first end of the storage container and the storage container has at a second end opposite the first end a divergent portion facilitating holding the storage container and beyond the opening in the upward direction of the diffusing apparatus so as to divert the flow of air exiting the opening to a periphery of the diffusing apparatus.

This can further tend to disperse the air charged with the evaporated substance into the ambient air.

In accordance with one embodiment the divergent portion is flush with the upper wall of the casing or beyond the upper wall in the downward direction of the diffusing apparatus, that is to say for example below the upper wall of the casing, and the divergent portion is shaped so as to divert the flow of air leaving the opening toward a periphery of the diffusing apparatus.

In accordance with one embodiment the casing has a lower end opposite the upper wall of the casing and this lower end has a plane surface. Thus the diffusing apparatus can be placed on a plane horizontal surface, for example on the surface of a table or more generally of an item of furniture.

Various positions are possible for the air intake. In accordance with one embodiment the air intake is on the lateral wall of the casing. In accordance with one embodiment the air intake is beyond the porous body in the downward direction of the diffusing apparatus, that is to say for example below the porous body in the position of use. In particular the casing has a lower end opposite the upper wall of the casing and beyond the porous body in the downward direction of the diffusing apparatus, that is to say for example below the porous body in the position of use, and the air intake is situated at this lower end.

In accordance with one embodiment the fan is downstream of the air intake and upstream of the porous body in a direction of circulation of the flow of air. Thus the substance evaporated at the level of the evaporation surface does not pass through the fan.

Various positions are possible for the fan. In accordance with one embodiment the fan is disposed beyond the porous body in the downward direction of the diffusing apparatus, that is to say for example below the porous body in the position of use. In accordance with one embodiment the fan is disposed between the lateral wall of the casing and the internal wall of the casing.

In accordance with one embodiment the fan is an axial fan or a centrifugal fan.

In accordance with one embodiment the aeration system further comprises an air guide member, the air guide member including at least one through-hole facing the evaporation surface.

In accordance with one embodiment the air guide member is disposed under the porous body in the position of use.

In accordance with one embodiment the air guide member includes a plurality of through-holes facing the evaporation surface.

In accordance with one embodiment the through-holes have a circular or oval section with an inside diameter between 0.1 mm and 4 mm inclusive.

In accordance with one embodiment the heating member comprises an electrical resistance placed directly on an exterior surface of the porous body or received in part in an opening, preferably a blind opening, in the porous body.

In accordance with one embodiment the heating member extends through the air guide member.

In accordance with one embodiment the heating member and/or the air guide member include(s) a depression situated beyond a lower surface of the hollow body in the downward direction of the diffusing apparatus, that is to say for example below the lower surface of the porous body in the position of use, the depression having a concavity facing toward the porous body, for example facing toward the lower surface of the porous body.

With this kind of depression if dripping of the substance in the liquid state occurs the substance that has dripped in this way accumulates in the depression.

In accordance with one embodiment the diffusing apparatus further comprises an electronic circuit card.

In accordance with one embodiment the diffusing apparatus further comprises a control device configured to control the heating member as a function of a setpoint temperature in the porous body.

In accordance with one embodiment the control device is on the electronic circuit card.

In accordance with one embodiment the control device is further configured to control an operating speed of the fan.

In accordance with one embodiment said substance includes at least one compound selected from semiochemical molecules, pheromones, allomones, kairomones, synomones of natural or synthetic origin.

In accordance with one embodiment the substance is a solution containing at least one sexual or non-sexual pheromone, an allomone, a synomone or a kairomone intended to cause a positive or negative response in the target species, the behavioral result of which may be sexual confusion, confusion of some other kind, sexual attraction, attraction of some other kind, repulsion of any kind, in arthropods, including arachnids, or hexapods, including in particular insects, including harmful insects.

In accordance with one embodiment the substance is a solution containing at least one pheromone or one sexual pheromone, an allomone, a synomone or a kairomone intended to provoke a positive or negative result in the target species, the behavioral result of which may be in particular pacification, relaxation, euphoria or intimidation in the classes of mammalia and birds.

In accordance with one embodiment the substance contains a solvent chosen from isopropyl myristate, glycol dipropylene, monomethyl glycol dipropylene and an isoparaffin hydrocarbon, for example an L or P or N or V isoparaffin.

In accordance with one embodiment the substance includes at least one compound selected from the group formed by odoriferous agents usable on man or on animals, semiochemical substances, cosmetic agents, essential oils, perfumes, disinfectants, odor neutralizing agents and phytosanitary and agricultural agents. In one embodiment the substance is a solution containing at least one compound selected from the above group.

In accordance with one embodiment the substance includes at least one compound selected from the group formed by odoriferous agents usable on man or on animals, semiochemical substances, cosmetic agents, essential oils, perfumes, disinfectants and phytosanitary and agricultural agents. In accordance with one embodiment the substance is a solution including at least one compound selected from the above group.

In accordance with one embodiment the substance contains at least one compound selected from the group formed by odoriferous agents usable on man, cosmetic agents, essential oils, perfumes, disinfectants, odor neutralizing agents. In accordance with one embodiment the substance is a solution including at least one compound selected from the above group.

In accordance with one embodiment the substance includes at least one compound selected from the group formed by odoriferous agents usable on man, cosmetic agents, essential oils, perfumes, disinfectants. In accordance with one embodiment the substance is a solution including at least one compound selected from the above group.

In accordance with one embodiment the odoriferous agents usable on animals are selected from fatty acids or esterified forms of fatty acids such as methyl oleate, methyl palmitate, dimethyl azelate and dimethyl pimelate.

In accordance with one embodiment the substance in the liquid state has a viscosity greater than 1 cPa·s at 25° C., for example greater than 8 cPa·s at 25° C., and less than 1 cPa·s at 60° C.

In accordance with one embodiment the substance has at atmospheric pressure a boiling point between 30° C. and 400° C. inclusive.

In accordance with one embodiment the substance is in the liquid state at ambient temperature and said storage container further contains an internal cellular retention member impregnated with said substance in the liquid state.

In accordance with one embodiment the cellular retention member comprises a material selected from felt, for example wool felt, and a melamine foam.

In accordance with one embodiment a plurality of cellular retention members are disposed in the storage container and in contact.

In accordance with one embodiment the storage container has no opening other than the drain orifice and the storage container contains in addition to the substance a gas phase occupying at least 20% of the volume of the storage container.

The first temperature may be fixed in different ranges. If the diffusing apparatus is intended to be used in the open air the first temperature will in particular be chosen as a function of local climate data. In accordance with some embodiments the first temperature is for example between 1° C. and 50° C. inclusive, or between 5° C. and 40° C. inclusive, or between 10° C. and 35° C. inclusive, or between 15° C. and 25° C. inclusive.

In accordance with one embodiment the substance has a viscosity varying as a function of temperature, said viscosity being such that in the position of use the substance is not able to flow through the porous body at any ambient temperature below a first temperature, the first temperature being above 0° C., and the substance flows through the porous body at a second temperature above the first temperature.

In accordance with one embodiment the substance is in the liquid state at ambient temperature. For example the substance may have at atmospheric pressure a melting point between −70° C. and 0° C. inclusive.

In accordance with one embodiment the substance is in the solid state at ambient temperature. For example the substance may have at atmospheric pressure a melting point above 30° C., for example between 30° C. and 40° C. inclusive.

In accordance with one embodiment the diffusing apparatus includes a plurality of storage containers for containing different substances, each of the storage containers having a drain orifice and being received in the receptacle so that the drain orifice is oriented in a downward direction of the diffusing apparatus, that is to say oriented downward in the position of use. Such storage containers may be produced in different ways, for example with interior partitioning of a single reservoir or by means of a plurality of distinct reservoirs.

The invention also concerns use of a diffusing apparatus as described above for dispersing into the air in the vapor state a substance in the liquid or solid state at ambient temperature in which the diffusing apparatus is in the position of use, the downward direction of the diffusing apparatus being toward the bottom relative to the acceleration due to gravity.

In accordance with one embodiment the diffusing apparatus is situated in a closed place, such as a greenhouse or a building, or in a place sheltered from precipitation.

### BRIEF DESCRIPTION OF THE FIGURES

The invention will be better understood and other aims, details, features and advantages thereof will become more clearly apparent in the course of the following description of particular embodiments of the invention provided by way of non-limiting illustration only and with reference to the appended drawings.

FIG. 1 is a perspective view of a diffusing apparatus in accordance with a first variant.

FIG. 2 is another perspective view of the diffusing apparatus from FIG. 1 in which a removable assembly has been received in the receptacle of the diffusing apparatus.

FIG. 3A is a perspective view of the removable assembly intended to be received in the receptacle of the diffusing apparatus.

FIG. 3B is another perspective view of the removable assembly from FIG. 3A.

FIG. 4A is a view of the storage container of the removable assembly from FIGS. 3A and 3B in section taken along the line IV-IV in FIG. 3A.

FIG. 4B is a view in section analogous to FIG. 4A showing the storage container and the dispensing member of the removable assembly from FIGS. 3A and 3B.

FIG. 4C is a view to a larger scale partially in perspective and partially in section of the detail IVC from FIG. 4B showing a variant of the removable assembly.

FIG. 5A is a view in section taken along the line V-V in FIG. 2.

FIG. 5B is another view in perspective and in section taken along the line V-V in FIG. 2.

FIG. 5C is a view in section analogous to FIG. 5A showing one possible way of using the diffusing apparatus.

FIG. 6 is a perspective view of a diffusing apparatus in accordance with a second variant.

FIG. 7 is a view in perspective and in section taken along the line VII-VII in FIG. 6.

FIG. 8 is a view in section of a diffusing apparatus in accordance with a third variant.

FIG. 9 is a schematic view in section of a diffusing apparatus in accordance with a fourth variant.

FIG. 10 is a schematic view in section of a diffusing apparatus in accordance with a fifth variant.

FIG. 11 is a schematic view in section of a variant of the heating member of the diffusing apparatus.

### DESCRIPTION OF EMBODIMENTS

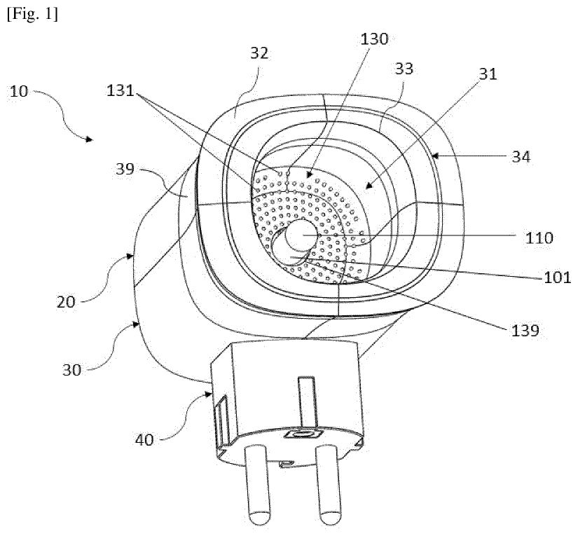

FIG. 1 is a perspective view of a first variant of a diffusing apparatus intended to disperse into the air in the vapor state a substance in the liquid state at ambient temperature. This diffusing apparatus bears the reference 10 in the figures; for convenience it is referred to hereinafter as “the apparatus 10”.

The apparatus 10 includes a fixed part generally designated 20.

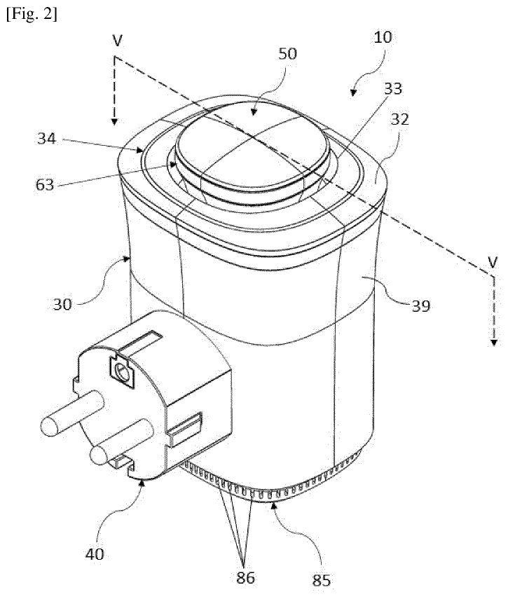

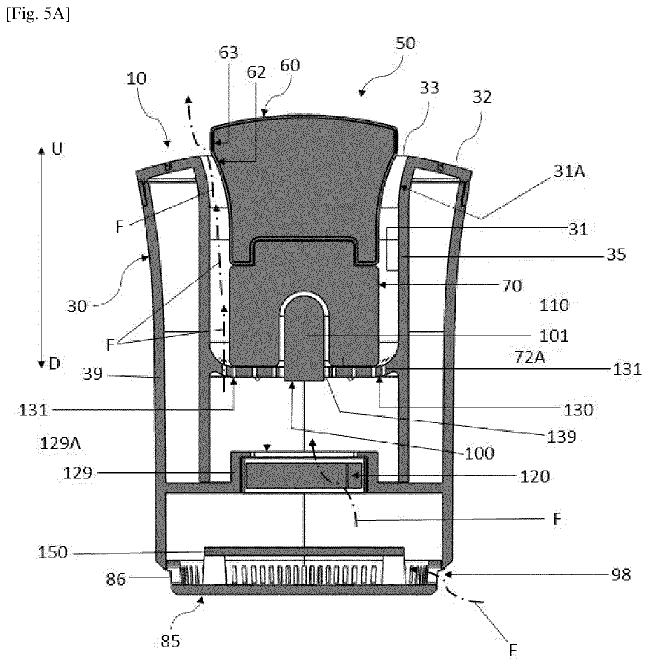

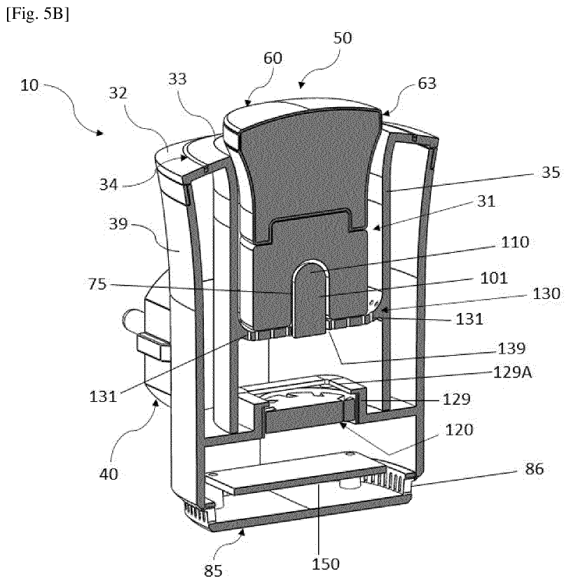

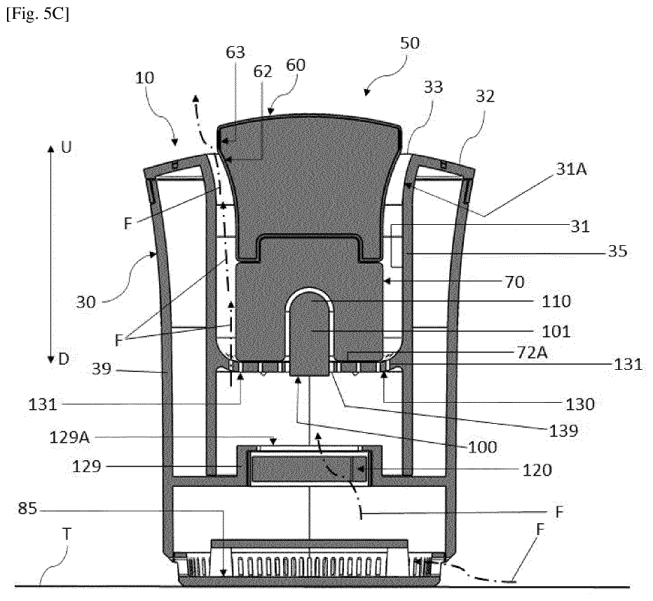

The fixed part 20 is described first. The fixed part 20 comprises a casing 30 and an electrical plug 40. FIGS. 5A, 5B and 5C are views in section of the apparatus 10 taken along the line V-V in FIG. 2 and therefore reveal components of the apparatus 10 disposed inside the casing 30.

The casing 30 defines a receptacle 31. The receptacle 31 is intended to receive a removable assembly 50 described hereinafter. Here the receptacle 31 has a section the shape of a rounded square but it could equally well have a circular, elliptical or even polygonal section. The receptacle 31 discharges onto an upper wall 32 of the casing 30 via an opening 33. The receptacle 31 is defined by an internal wall 35 (cf. FIG. 5A, FIG. 5B and FIG. 5C) of the casing 30 that extends from the upper wall 32 to the interior of the casing 30. The reference 39 in the figures designates an external lateral wall of the casing 30.

The electrical plug 40 is fixed to the casing 30 in any appropriate manner. In the example represented here the electrical plug 40 is a mains electrical plug. The apparatus 10 can therefore be supplied with electricity from the mains, which in particular enables use in a closed space such as a greenhouse or building, for example, or in a place sheltered from precipitation. Nevertheless, other types of electrical plug 40 can be envisaged, in particular direct current electrical plugs.

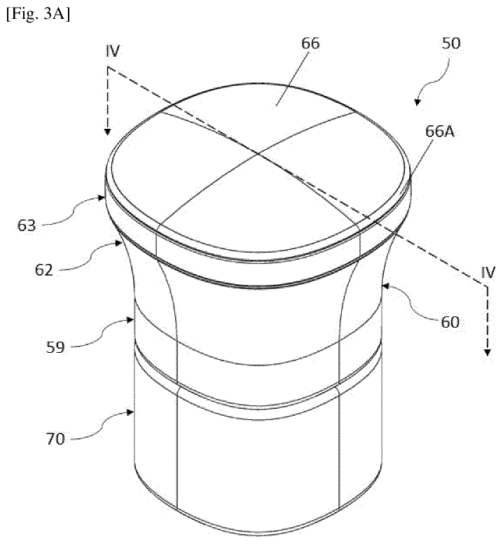

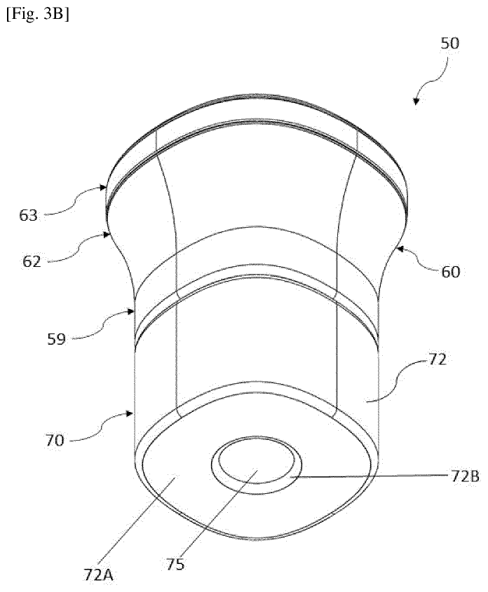

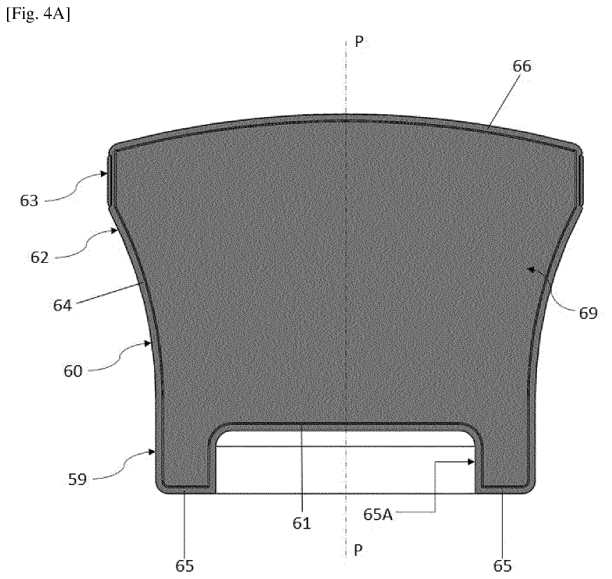

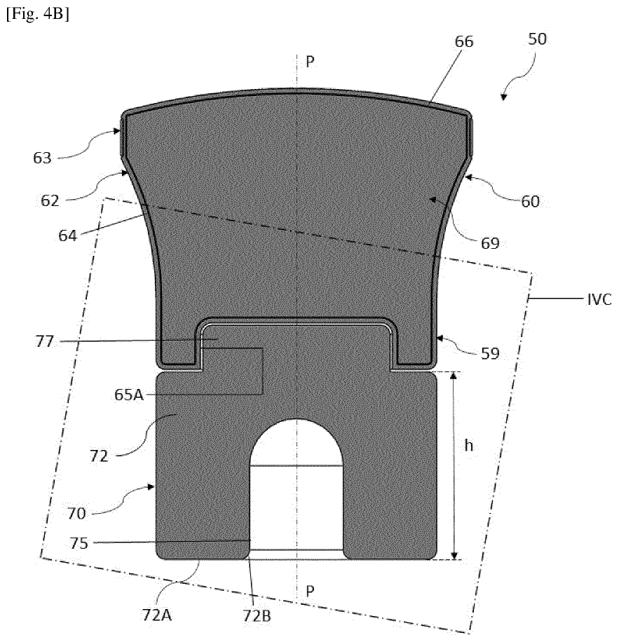

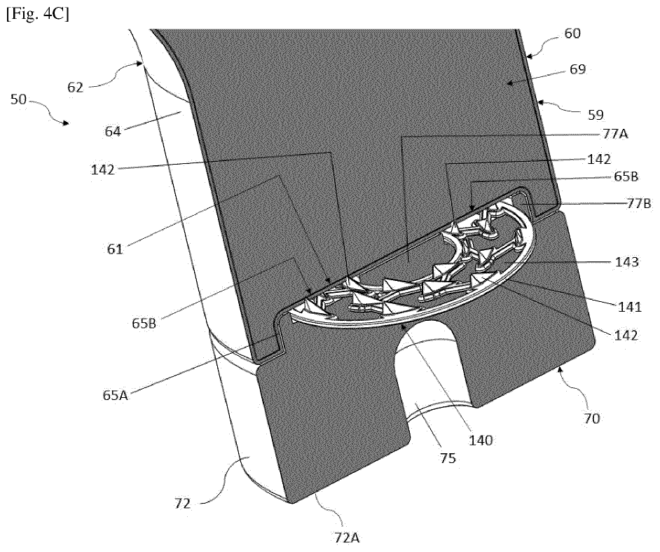

The removable assembly 50 is described next with reference to FIGS. 3A, 3B, 4A and 4B. The removable assembly 50 comprises a storage container 60 and a wick 70. The wick 70 is fastened to the storage container 60 so that the removable assembly 50 can be inserted in one piece in the receptacle 31 by picking up the removable assembly 50.

FIG. 4A is a view in section of the storage container 60 alone. As can be seen better in FIG. 4A a lower portion 59 of the storage container 60 has a right cylinder section the directrix of which is a main axis P-P and the generatrix of which is here in the shape of a rounded square. In other words, a section of the storage container 60 perpendicular to the axis P-P at the level of the lower portion 59 is in the shape of a rounded square. This section is preferably identical to the section of the receptacle 31. The interior volume 69 of the storage container 60 is delimited by a peripheral wall 64, an upper wall 66 and a lower wall 65.

As can be seen better in FIG. 4A the lower wall 65 includes a recess 65A. A drain orifice 61 is made in all or part of the bottom of a recess 65A. The drain orifice 61 is oriented downward in a position of use of the removable assembly 50, this position of use being represented in FIG. 2, FIG. 5A, FIG. 5B and FIG. 5C.

The terms “downward”, “lower” and “upper” are to be understood relative to the acceleration due to gravity in the position of use.

Likewise the terms “below” and “above” are to be understood relative to the acceleration due to gravity in the position of use.

In an equivalent way the apparatus 10 may be assigned a downward direction D and an upward direction U (cf. FIG. 5A and FIG. 5C), the downward direction D and the upward direction U being opposite. In the position of use, relative to the acceleration due to gravity the downward direction D is downward and the upward direction U is upward; to be more precise, the downward direction D is parallel to the acceleration due to gravity and oriented toward the ground and the upward direction U is opposite the downward direction D. The terms “downward”, “lower” and “below” are then to be understood in the downward direction D; the terms “upper” and “above” are then to be understood in the upward direction U.

In the position of use the axis P-P may be parallel to the acceleration due to gravity and therefore to the downward direction D and the upward direction U.

The storage container 60 may exhibit circular symmetry about the axis P-P.

In one embodiment the storage container 60 is made by injection molding an appropriate plastic material such as polypropylene (PP) for example. One or more pressure vents (not represented) may be formed in the upper wall 66 and/or the peripheral wall 64 in order to guarantee that the liquid substance contained in the storage container 60 can continue to flow as the storage container 60 empties. Alternatively, the storage container 60 has no opening other than the drain orifice 61 and contains in addition to the liquid substance a gas phase occupying at least 20% of the volume of the storage container 60.

An intermediate portion 62 of the storage container 60 is divergent, that is to say shaped so that the peripheral wall 64 diverges from the main axis P-P in the direction away from the lower portion 59. This intermediate portion 62 is extended by a terminal portion 63 at the level of which the peripheral wall 64 is again parallel to the main axis P-P. Clearly the intermediate portion 62 and the terminal portion 63 facilitate picking up of the storage container 60 by a user, because the latter can easily take hold of the storage container 60 by placing their fingers around the terminal portion 63 and then placing the tips of their fingers on the intermediate portion 62.

The interior volume 69 of the storage container 60 can optionally be at least in part filled with a polymer foam (not represented) impregnated with the liquid substance. Alternatively the interior volume 69 may simply be filled with the liquid substance.

In certain variants that are not represented the interior volume 69 can contain a plurality of different liquid substances. To that end the interior volume 69 may be partitioned so as to define a plurality of interior sub-volumes, each interior volume 69 containing a liquid substance and including a drain orifice 61 for that liquid substance. In accordance with other variants that are not represented the removable assembly 50 may comprise a plurality of storage containers 60, possibly identical, each containing a liquid substance and all connected to the wick 70 described next. These variants enable a removable assembly 50 to be provided containing a plurality of liquid substances that do not mix until the moment at which they are diffused by the apparatus 10.

It is to be noted that the upper wall 66 may be provided by a lid that is removable to enable the interior volume 69 of the storage container 60 to be re-filled when it has been emptied of liquid substance. The reference 66A in FIG. 3A designates a chamfer that can make it possible to facilitate the removal of such a lid.

The storage container 60 and a wick 70 are represented in section in FIG. 4B.

The wick 70 is positioned at the outlet of the drain orifice 61 so that the liquid substance is able to pass from the interior space 69 of the storage container 60 to the wick 70 and from there to evaporation surfaces consisting of the exterior walls of the wick 70. Here the wick 70 includes a main portion 72 with the same section as the lower portion 59 of the storage container 60. Furthermore, an opening 75 extends in the wick 70, here parallel to the axis P-P, from an interior surface 72A of the main portion 72. The opening 75 is preferably a blind opening as represented in the figures.

The wick 70 may exhibit circular symmetry about the axis P-P.

The wick 70 may be attached to the storage container 60 in various ways. In the example represented in the figures the wick 70 includes a spigot 77 intended to be received in the recess 65A, for example forced into the recess 65A.

The wick 70 is made partially or entirely of a porous material, for example wood, textile, ceramic or polymer. Another example of a possible porous material is a porous metal obtained by sintering a metal powder or a metal alloy powder. Such porous metals are known as such and techniques for obtaining them are not described in detail here.

As already mentioned hereinabove the storage container 60 includes a drain orifice 61 that is oriented downward in a position of use of the removable assembly 50. The wick 70 is positioned at the outlet of this drain orifice 61 so as to receive and because of its porosity to be impregnated with the liquid substance contained in the interior volume 69 of the storage container 60.

FIG. 4C is a view to a larger scale of the detail IVC from FIG. 4B and thus shows the connection between the wick 70 and the drain orifice 61 in one particular embodiment of the removable assembly 50. In the embodiment represented here the drain orifice 61 is blocked by a foil 65B. The wick 70 includes a central spigot 77A. The removable assembly 50 further includes a perforator device 140 disposed between the wick 70 and the drain orifice 61. The perforator device 140 includes an annular main body 141 fitted onto the central spigot 77A of the wick 70 and optionally retained in position relative to the central spigot 77A by an annular projection 77B on the wick 70 around the central spigot 77A. The main body 141 includes a plurality of teeth 142 and between the teeth 142 a plurality of gaps 143. The teeth 142 are disposed so as to be able to perforate the foil 65B. Once the foil 65B has been perforated by the teeth 142 the liquid substance flows through the holes produced by the teeth 142 and then through the gaps 143 and comes to wet the spigot 77 and then the rest of the wick 70 by capillarity. This perforation of the foil 65B by the teeth 142 can in particular be obtained by moving the wick 70 in the direction of the drain orifice 61. The receptacle 31 and the wick 70 may be shaped so that this movement of the wick 70 in the direction of the drain orifice 61 occurs during installation of the removable assembly 50 in the receptacle 31.

The perforator device 140 may for example be produced by injection molding an appropriate polymer. Alternatively, it could equally well be a metal part, for example a casting, or a ceramic part. Furthermore, the perforator device 140 could consist of a plurality of annular sectors, possibly but not necessarily fastened to one another.

The teeth 142 and the gaps 143 are preferably regularly spaced so as to favor uniform impregnation of the wick 70 by the liquid substance.

The geometry of the teeth 142 and the gaps 143 represented in FIG. 4C is merely one example. Numerous other configurations are possible. Furthermore, in addition to or instead of the teeth 142 hollow or solid needles may be disposed on the perforator device 140 so as to perforate the foil 65B as has just been described.

The connection that has just been described between the wick 70 and the interior volume 69 of the storage container 60 via the drain orifice 61 is merely an example. Numerous other connections are possible. In particular if the interior volume 69 of the storage container 60 is at least in part a polymer foam impregnated with the liquid substance the wick 70 may include one or more spigots that pass through the drain orifice 61 until it or they come(s) into contact with that polymer foam. One or more seals (not represented) may then be disposed so as to guarantee a sealed connection between the wick 70 and the drain orifice 61.

In accordance with another variant that is not represented the spigot 77 may be configured so that the wick 70 can be screwed into the drain orifice 61 of the storage container 60. In this case the spigot 77 may optionally include microneedles (not represented) that perforate the foil 65B when screwing in the wick.

As mentioned hereinabove the removable assembly 50 is intended to be received in the receptacle 31 defined by the casing 30. FIG. 2, FIG. 5A, FIG. 5B and FIG. 5C represent the removable assembly 50 in position in the receptacle 31 in the position of use. As represented in these figures, in the position of use the terminal portion 63 projects relative to the upper wall 32 of the casing 30. In an equivalent manner the terminal portion 63 is above the opening 33, that is to say beyond the opening 33 in the upward direction U. This facilitates holding the storage container 60 and replacing the removable assembly 50. Although this is not represented in the drawings, the terminal portion 63 could even have a greater outside dimension approximately equal to or even greater than the greatest dimension on the opening 33 of the receptacle 31. Alternatively, the terminal portion 63 can be flush with the upper wall 32 or below the upper wall 32, that is to say beyond the upper wall 32 in the downward direction D.

Referring again to FIG. 1, there is seen the bottom of the receptacle 31 as seen from above from the upper side of the apparatus 10. A heating member generally designated by the reference 100 and partially represented in the views in section in FIGS. 5A, 5B and 5C is disposed in the diffusion casing 30. The heating member 100 includes a rod 101 carrying at its upper end an electrical resistance 110. The rod 101 can extend through the opening 75 so that the electrical resistance 110 comes into contact with the walls of the opening 75 when the removable assembly 50 is in place in the receptable 31. By supplying the electrical resistance 110 with electricity or not it is possible to control the flow of the liquid substance through the wick 70 in accordance with the physical principles described in the documents WO 2019/243734 A1 and WO 2020/254733 A1.

The lower surface 72A of the wick 70 may be connected to the opening 75 by an inclined surface 72B (cf. FIG. 3B and FIG. 4B) so as to facilitate the insertion of the rod 101 and the electrical resistance 110 in the opening 75.

Alternatively, the wick 70 need not include the opening 75; the electrical resistance 110 is then placed directly on an exterior surface of the wick 70, for example the lower surface 72A.

The heating member 100 may include an electronic circuit card 150 (represented schematically in FIG. 5A, FIG. 5B and FIG. 5C) providing the electrical power supply to and control of the electrical resistance 110. For example a control device (not represented) such as a microprocessor may be located on the electronic circuit card 150 in order to control the electrical resistance 110 as a function of a setpoint temperature of the wick 70. This setpoint temperature can be measured by a temperature sensor (not represented) carried for example by the rod 101. Here the electrical circuit card 150 is carried by the lid part 85 described below, but may alternatively be disposed at various locations in the casing 30.

The apparatus 10 further includes an aeration system. This aeration system includes an air intake 98 and a fan 120. The air intake 98 is below the removable assembly 50 in the position of use. The fan 120, which can be seen in the views in section in FIGS. 5A, 5B and 5C, is below the removable assembly 50 in the position of use. It is clear that in this way the fan 120 directs a flow of air from the air intake 98 to the evaporation surfaces consisting of the exterior walls of the wick 70 and then to the opening 33. The chain-dotted line arrows F in FIG. 5A indicate the sense and the direction of this flow of air. Thus when the fan 120 and the electrical resistance 110 are functioning the apparatus 10 generates a flow of air charged with evaporated substance, this flow of air escaping via the opening 33. In other words the opening 33 forms the single air outlet of the aeration system of the apparatus 10.

Referring more particularly to FIG. 5A it is seen that, facing the intermediate portion 62 of the storage container 60, the receptacle 31 may have a divergent portion 31A at the level of which the walls of the receptacle 31 are curved so as to increase the section of the receptacle 31. For example the walls of the receptacle 31 at the level of the divergent portion 31A can have a curvature identical or substantially identical to the curvature of the intermediate portion 62. The curvatures of the divergent portion 31A and the intermediate portion 62 tend to divert the flow of air in the vicinity of the opening 33, as represented by the arrows F. This can tend to increase the dispersion of air charged with evaporated substance into the ambient air.

The air intake 98 may be produced in various ways. In the example represented here the lower end of the casing 30 is closed by a lid part 85 (cf. FIGS. 2, 5A, 5B and 5C). The lid part 85 features a plurality of openings 86 constituting the air intake 98. The openings 86 can optionally extend corresponding openings (not represented) in the casing 30 in line with the lid part 85. The lid part 85 may be produced in one piece with the casing 30 or fixed to the casing 30. Alternatively, the lid part 85 can be removably fixed to the casing 30. In this case in particular the lower end of the casing 30 can be connected to an air channel or another air intake; and the fan 120 can be located in this channel or air intake.

In the example represented here the lower end of the receptacle 31 is formed by an air guide part 130 (cf. FIG. 1, FIG. 5A, FIG. 5B and FIG. 5C) which is a bottom wall in particular protecting the fan 120 and the electrical components from access by the user. Here the air guide part 130 includes a plurality of through-holes 131 that allow the flow of air created by the fan 120 to pass through them. The air guide part 130 further includes a central through-orifice 139 for the rod 101 and the heating resistance 110 to pass through. The rod 101 may be fastened to the air guide part 130.

Here the air guide part 130 is flared but could instead be plane. It is further specified that although the drawings represent the lower surface 72A of the wick 70 as being almost in contact with the air guide part 130 a gap of the order of a few millimeters or a few centimeters for example may be provided between the lower surface 72A and the air guide part 130 so as to allow evaporation of the liquid substance at the level of the lower surface 72A.

The number, disposition and relative orientations of the through-holes 131 may be chosen in various ways to orient the flow of air over the exterior walls of the wick 70 and thereby to favor the evaporation of the substance on the exterior walls of the wick 70. Furthermore, although the figures show that the air guide part 130 includes a large number of through-holes 131 of circular or oval section with small dimensions (an inside diameter of the order of 0.1 mm to 4 mm for example), other dimensions and shapes of the through-holes are possible. For example, in a variant that is not represented the air guide part 130 includes in addition to or instead of the through-holes 131 an annular or circular through-orifice. In this variant the air guide part 130 can carry the rod 101; the annular or circular through-orifice may be under the rod 101.

Various constructions may be adopted for the fan 120. In the example represented here the fan 120 is an axial flow fan. Here the fan 120 is held in place in an internal depression 129 in the casing 30 under the heating member 100 and the air guide part 130. The bottom of this internal depression 129 includes a through-orifice 129A disposed between the wick 70 and the fan 120 to allow the flow of air to pass through it. Alternatively the fan 120 may be held in place in the casing 30 differently. Furthermore, the fan 120 may alternatively be a centrifugal fan or a fan of other type.

An air filter (not represented) may be disposed between the air intake 98 and the fan 120 in order to limit the risk of soiling of the wick 70 by undesirable particles from the outside.

Although the figures show that the air intake 98 is at the lower end of the casing 30, below the fan 120, which is itself below the wick 70, numerous other positions of the air intake 98 and the fan 120 in the casing 30 are possible.

In particular, the air intake 98 may be disposed on the exterior lateral wall 39 of the casing 30. In this case the air intake 98 is possibly but not necessarily farther down the upper wall 32 than the wick 70, that is to say beyond the wick 70 in the downward direction D. The fan 120 may be below the wick 70 as represented in the figures or between the exterior lateral wall 39 and the internal wall 35 defining the receptacle 31. Of course, in the latter case it is possible for through-holes analogous to the through-holes 131 in the air guide part 130 to be provided in the internal wall 35, facing the wick 70. In this latter case the fan 120 is possibly but not necessarily also farther down the upper wall 32 than the wick 70, that is to say beyond the wick 70 in the downward direction D.

The fan 120 may be controlled by the control device mentioned hereinabove.

The removable assembly 50 may be held in place in the receptacle 31 in various ways provided that the removable assembly 50 can be removed from the receptacle 31 in order to be replaced by an identical assembly when the liquid substance contained in the storage container 60 has all been used. For example, the removable assembly 50 may be screwed or clipped into the receptacle 31. In one embodiment the removable assembly 50 is limited to the container 60 while the wick 70 remains permanently in the apparatus 10. In another embodiment the wick 70 may be demounted and replaced independently of the container 60.

The casing 30 may be provided with an indicator light 34 indicating for example the functional status of the apparatus 10 as a function of control input from the control device mentioned hereinabove. In the example represented in the figures this indicator light 34 takes the form of a translucent ring illuminated by one or more colored light-emitting diodes and extending on the upper wall 32 of the casing around the opening 33. However, numerous other embodiments of the indicator light 34 are possible.

FIG. 5C shows that one possible way of using the apparatus 10 consists in placing its lower end on a plane and horizontal surface T. A surface is “horizontal” when it is perpendicular to the acceleration due to gravity. The surface T may be the surface of a table or more generally of an item of furniture. To enable the lower end of the apparatus 10 to be placed on the surface T the lower surface of the lid part 85 is plane, as represented in the figures.

### EXAMPLES

Two examples of the diffusing apparatus 10 as described hereinabove were made, identical apart from the construction of the wick 70. The two different wicks 70 of the two examples of the diffusing apparatus 10, hereinafter referred to respectively as “system 1” and “system 2”, have the following features:
system 1: the wick 70 is made of sintered alumina and has a pore size of 120 nm and a uniform porosity of 40%. The height h (cf. FIG. 4B) of the wick 70 is 8 mm;
system 2: the wick 70 is made of sintered alumina and has a pore size of 120 nm and a uniform porosity of 40%. The height h (cf. FIG. 4B) of the wick 70 is 25 mm.
Three experiments hereinafter referred to as “experiment 1”, “experiment 2” and “experiment 3” were conducted using system 1 or system 2.

In these three experiments a diffusion rate also referred to an evaporation rate is measured. These rates are measured in accordance with a gravimetric measurement protocol that consists in measuring the relative loss of mass of the system over time by considering (by visual inspection) that any loss of mass is the result of evaporation followed by expulsion of the molecules out of the system, excluding any leakage of liquid.

Some of the essential oils or food fragrances used in the experiments described hereinafter were diluted beforehand in dipropylene glycol (DPG), a substance having at atmospheric pressure a melting point of −39° C. and a boiling point of 230° C. The dilution in DPG is expressed in weight percent with the following convention: a mixture of 25 wt % rhubarb essential oil and DPG contains 25 wt % of rhubarb essential oil in DPG. One way of preparing such a mixture therefore consists in adding 75 g of DPG to 25 g of rhubarb essential oil.

### Experiment 1

Experimental device: Three different substances were successively diffused using system 1: rhubarb essential oil, eucalyptus pine essential oil, rose essential oil, the storage container 60 being filled each time with 5 g of essential oil, the latter not being diluted by addition of a solvent. Each time system 1 was allowed to function at an ambient temperature of 20° C. with the wick 70 maintained at a temperature of 35° C. and operating the fan 120 so as create a constant flow of air at 4 m³/h.

Result: The three substances tested flow through the wick 70 and evaporate, with no recondensation observed, with variable rates of diffusion. The loss of weight is linear over time, in contrast to what is observed with passive diffusers for which the diffusion rate is a function of concentration. The olfactory sensation is extremely powerful. The diffusion rates measured with each substance are set out in table 1 below.

TABLE 1

| | Undiluted | Undiluted | |
|---|---|---|---|
| | rhubarb | eucalyptus pine | Undiluted rose |
| Substance | essential oil | essential oil | essential oil |
| Diffusion rate in | 0.8 | 1.6 | 0.9 |
| gram/day | | | |

### Experiment 2

Experimental device: Three different substances were successively diffused using system 1: undiluted eucalyptus pine essential oil, mixture of 25 wt % eucalyptus pine essential oil and DPG, mixture of 50 wt % eucalyptus pine essential oil and DPG, the storage container 60 being filled each time with 5 g total weight of substance. Each time the system 1 was allowed to function at an ambient temperature of 20° C. with the wick 70 maintained at a temperature of 35° C. and operating the fan 120 so as create a constant flow of air at 4 m³/h.

Result: The substances tested flow through the wick 70 and evaporate with different diffusion rates, which are set out in table 2 below. No recondensation was observed. The loss of weight is linear over time, in contrast to what is observed with passive diffusers for which the diffusion rate is a function of concentration. It has been found qualitatively that the olfactory sensation is less powerful with greater dilution with DPG. It has been observed qualitatively that the substances perfumed a 120 m² room in less than 15 minutes.

TABLE 2

| | Undiluted | Mixture of 25 wt % | Mixture of 50 wt % |
|---|---|---|---|
| | eucalyptus | eucalyptus pine | eucalyptus pine |
| | pine essential | essential oil and | essential oil and |
| Substance | oil | DPG | DPG |
| Diffusion | 1.66 | 1.0 | 0.5 |
| rate in | | | |
| gram/day | | | |

### Experiment 3

Experimental device: Five substances were successively diffused using system 2: mixture of 10 wt % marine odor essential oil and DPG, mixture of 50 wt % marine odor essential oil and DPG, undiluted marine odor essential oil, mixture of 50 wt % pièce montée food fragrance and DPG, mixture of 2 wt % lobster food fragrance and DPG, the storage container being filled each time with 15 g total weight of substance. Each time system 2 was allowed to function at an ambient temperature of 20° C. with the wick 70 maintained at a temperature of 32° C. and operating the fan 120 so as to create a constant flow of air at 4 m³/h. The diffusion rates measured with each food fragrance are set out in table 1 below.

Result: The substances tested flow through the wick 70 and evaporate with different diffusion rates, which are set out in table 3 below (in table 3 the abbreviation “e.o.” means essential oil and “f.f.” means food fragrance). The loss of weight is linear over time. The quality of the olfactory sensation is excellent and constant over time.

TABLE 3

| | Mixture of | Mixture of | | Mixture of | Mixture of |
|---|---|---|---|---|---|
| | 10 wt % | 50 wt % | | 50 wt % pièce | 2 wt % |
| | marine e.o. | marine e.o. | Undiluted | montée f.f. | lobster f.f. |
| Substance | and DPG | and DPG | marine e.o. | and DPG | and DPG |
| Diffusion | 6.2 | 5.5 | 5.2 | 4.0 | 29.2 |
| rate in | | | | | |
| gram/day | | | | | |

### Variants

The construction of the apparatus 10 represented in FIGS. 1 to 5C is only an example. Numerous other constructions may be envisaged.

In accordance with variants that are not represented the opening 33 may form one of the air outlets of the aeration system of the apparatus 10, one or more additional air outlets being formed in the casing 30 in fluid communication with the receptacle 31. In this case some of the air charged with evaporated substance is directed by the aeration system to these additional air outlets instead of being directed to the opening 33.

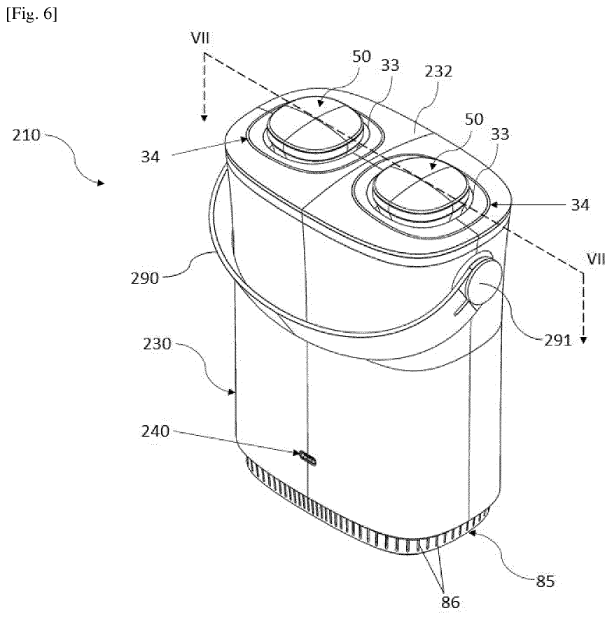

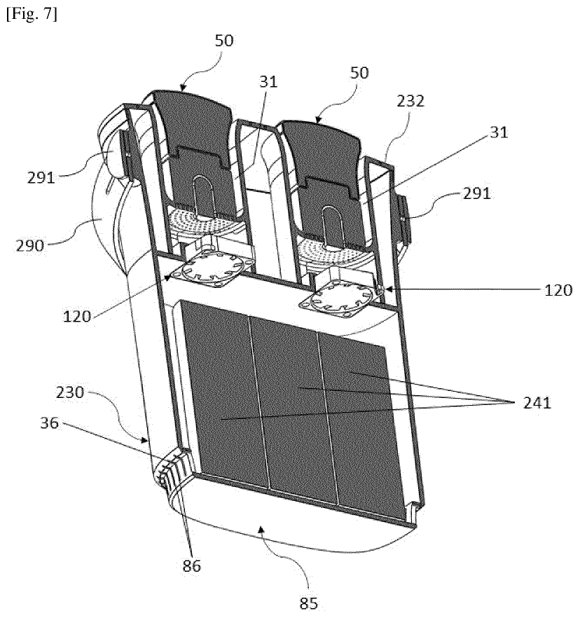

A second variant apparatus 210 is represented in perspective in FIG. 6 and in section in FIG. 7. In these figures elements identical to those described above with reference to FIGS. 1 to 5C bear identical references and are not described in detail again. The apparatus 210 is distinguished from the apparatus 10 in that the casing 230 includes two receptacles 31 the openings 33 of which both discharge onto the upper wall 232 of the casing 230. Each of the two receptacles 31 receives a removable assembly 50. Thus the apparatus 210 can be used to diffuse two different substances by inserting into the two receptacles 31 removable assemblies 50 containing different substances. Of course, a greater number of receptacles 31 and removable assemblies 50 may be provided with the same apparatus 210 if required. Here each of the receptacles 31 is associated with a fan 120 but alternatively a single fan 120 may be associated with all the receptacles 31.

It is also seen in FIG. 6 that the apparatus 210 is fed with electricity by a direct current plug 240, here of USB-C type, and it is seen in FIG. 7 that the apparatus 210 contains a plurality of batteries 241, for example of the lithium-ion type, disposed inside the casing 230. Thus the apparatus 210 is able to function even in the absence of a mains supply.

Furthermore, the apparatus 210 is provided with a carrying handle 290, here articulated on two sides of the casing 230 by two opposite articulations 291. The apparatus 210 can therefore be easily carried in the hand of a user.

The apparatus 210 may alternatively be supplied from the mains like the apparatus 10 and/or not include a carrying handle 290.

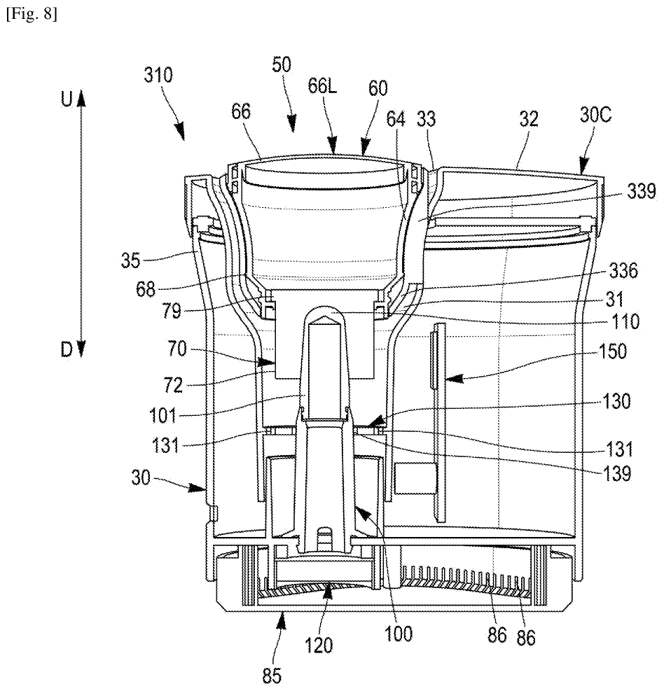

A third variant of an apparatus 310 is represented in section in FIG. 8. In this figure elements identical to those described above with reference to FIGS. 1 to 5C bear identical references and are not described in detail again.

The apparatus 310 is distinguished from the apparatus 10 in that the receptacle 31 includes an internal separator wall 336. The internal separator wall 336 is between the peripheral wall 64 of the storage container 60 and the internal wall 35 defining the receptacle 31.

The wick 70, or at least a lower part of its main portion 72, is not surrounded by the internal separator wall 336 whereas the peripheral wall 64 of the storage container 60 is entirely surrounded by the internal separator wall 336. Thus the flow of air charged with evaporated substance at the level of the wick 70 does not flow along the peripheral wall 64 but along the internal separator wall 336. Consequently, if recondensation of the evaporated substance occurs in the receptacle 31 recondensation does not occur on the peripheral wall 64 but on the internal separator wall 336. The risk of a user of the apparatus 310 accidentally touching the recondensed substance is therefore significantly reduced.

Alternatively, the peripheral wall 64 of the storage container 60 may be only partially surrounded by the internal separator wall 336; in this case it is nevertheless preferable for the surface of the peripheral wall 64 that is not surrounded by the internal separator wall 336 to be limited in order to limit recondensation of the evaporated substance on the peripheral wall 64.

The internal separator wall 336 is permanently in the casing 30. In the example represented here the internal separator wall 336 is connected to the internal wall 35 by one or more ribs 339, one of which can be seen in FIG. 8.

The upper wall 32 and the internal wall 35 are formed in an upper portion 30C of the casing 30. This upper portion 30C is mounted so that it is able to slide relative to the lateral wall 39 in the downward direction D. To be more precise the upper portion 30C is mounted so as to be able to slide between:
a raised position in which the upper wall 66 of the storage container 60 is beyond the upper wall 32 in the downward direction D when the storage container 60 is received in the receptacle 31;
a lowered position in which the upper wall 66 is beyond the upper wall 32 in the upward direction U when the storage container 60 is received in the receptacle 31. Thus when the upper portion 30C is in the raised position (represented in FIG. 8) the storage container 60 may be difficult or even impossible for a user of the apparatus 310 to access while sliding the upper portion 30C toward the lowered position renders the storage container 60 more easily accessible for the user. The upper portion 30C can therefore make the apparatus 310 safer to use.

The casing 30 may optionally further include a mechanical locking device, for example a ratchet device, to lock the upper portion 30C in the lowered position and/or a return device, for example a spring, to return the upper portion 30C to the raised position.

The rib or ribs 339 may be fastened to the internal wall 35 so that the internal separator wall 336 is fastened to the upper portion 30C and therefore slides with the upper portion 30C. Alternatively, the internal separator wall 336 need not slide with the upper portion 30C; the ribs 339 can then be received in notches (not represented) in the internal wall 35 having a clearance corresponding to the distance travelled by the upper portion 30C between the raised position and the lowered position.

Alternatively the upper portion 30C need not slide relative to the lateral wall 39.

The removable assembly 50 may be fixed to the casing 30 in the receptacle 31 via the internal separator wall 336.

In the example represented here the peripheral wall 64 of the storage container 60 includes a projection 68 and the projection 68 is received in a groove facing it in the internal separator wall 336. The projection 68 and the groove are shaped so that the cooperation of the projection 68 with the groove enables fixing of the storage container 60 by screwing or by a bayonet-type coupling for example. Of course, fixing in this way by screwing or by a bayonet-type coupling can be implemented in various ways, for example by means of a plurality of projections and corresponding grooves. The storage container 60 can also be fixed to the internal separator wall 336 in other ways, for example by clipping or snap-fastening it.

In any event, the storage container 60 is therefore fixed to the internal separator wall 336 while the wick 70 is already connected to the storage container 60. In other words the removable assembly 50 is inserted in one piece in the receptacle 31 and the storage container 60 is fixed to the internal separator wall 336 during insertion of the removable assembly 50 into the receptacle 31. The lower end of the internal separator wall 336 defines an opening enabling the wick 70 to reach the position represented in FIG. 8.

In the embodiment represented here the wick 70 includes a flange 79 with a greater diameter than the drain orifice 61 whereas the main portion 72 of the wick 70 has a section equal to or very slightly less than the section of the drain orifice 61. The wick 70 is inserted into the storage container 60 from an open upper end of the latter, after which the storage container 60 is filled with substance and closed by a lid 66L, the lid 66L defining the upper wall 66 of the storage container 60. Furthermore, a retaining element (not represented) may be provided to hold the wick 70 in place in the storage container 60 in the position represented in FIG. 8. In one embodiment the retaining element takes the form of a ring or an analogous part that is forced into the storage container 60 via its open upper end until the ring bears on the wick 70 in order to close the storage container 60 by means of the lid 66L. In another embodiment the retaining element is a spacer extending from the lid 66L to the wick 70 and one end of which bears on the wick 70 when the lid 66L closes the storage container. In a further embodiment the retaining element takes the form of a pleat, that is to say a projection formed on the peripheral wall 64 of the storage container 60 and projecting into the interior volume 69 of the storage container 60. Before closing the storage container 60 by means of the lid 66L the wick 70 is forced past the pleat. Once the wick 70 has passed the pleat the pleat opposes the wick 70 passing the pleat in the opposite direction and therefore holds the wick 70 in place. One or more seals (not represented) may be disposed in such a manner as to guarantee a sealed connection between the wick 70 and the drain orifice 61 and/or sealed closure of the lid 66L.

Alternatively, it is also possible to envisage that the various connections of the wick 70 to the storage container 60 described hereinabove are made in the apparatus 310.

The functioning of the apparatus 310 is identical to that of the apparatus 10 and of the apparatus 210 and is therefore not described in detail again.

It may be envisaged that the internal separator wall 336 is produced in the apparatus 10 or in the apparatus 210. It can also be envisaged that the upper portion 30C of the casing 30, whether sliding or not, be used in the apparatus 10 or in the apparatus 210. It may also be envisaged that the connection of the wick 70 to the storage container 60 by means of the flange 79 be used in the apparatus 10 or in the apparatus 210.

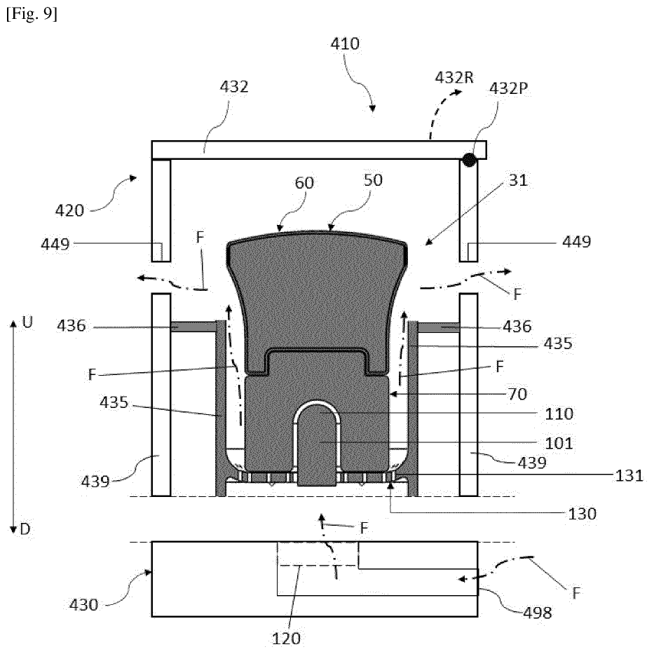

A fourth variant of an apparatus 410 is represented schematically in section in FIG. 9. In that figure elements identical to those described above with reference to FIGS. 1 to 5C bear identical references and are not described in detail again.

The fixed part 420 of the apparatus 410 includes a frame 430 that is partially represented in FIG. 9. The receptacle 31 is in a hollow portion of the frame 430, for example in a hollow portion at the upper end of the frame 430.

In a manner that is not represented the frame 430 may take a very large number of forms, for example the form of a column resting on the floor via a foot, a column anchored to the floor, etc.

In the embodiment represented here the receptacle 31 is at the upper end of the frame 430 and the receptacle 31 is delimited by a peripheral wall 439 of the frame 430, an upper wall 432 of the frame 430 and internal walls 435, 436 of the frame 430. The internal walls 435, 436 support or are formed in one piece with the air guide part 130. In a manner that is not represented the peripheral wall 439 may have a circular, rectangular or other horizontal section.

The upper wall 432 may pivot relative to the peripheral wall 439 by means of a pivot 432P so that the upper wall 432 can be pivoted (cf. 432R in FIG. 9) to uncover an upper opening delimited by the peripheral wall 439 and enabling insertion of the removable assembly 50 in the receptacle 31.

As represented schematically in FIG. 9 the frame 430 has an air intake 498 below the wick 70. For example the air intake 498 may be at a lower end of the frame 430. The fan 120 (represented schematically in dashed line in FIG. 9) is situated here in the frame 430 between the air intake 498 and the air guide part 130 in the downward direction D. Alternatively, the air intake 498 may be connected to an air channel or another air intake; and the fan 120 may be in that channel or air intake.

An opening 449 is formed in all or part of the peripheral wall 439 above the wick 70 in the upward direction U. For example, as represented here the opening 449 faces the storage container 60.

In operation the fan 120 directs a flow of air from the air intake 498 to the evaporation surfaces consisting of the exterior walls of the wick and then to the opening 449. The chain-dotted line arrows F in FIG. 9 indicate the sense and the direction of this flow of air. Thus when the fan 120 and the electrical resistance 110 are functioning the apparatus 410 generates a flow of air charged with evaporated substance exiting via the opening 449. In other words here the opening 449 forms the single air outlet of the aeration system of the apparatus 410.

In accordance with variants that are not represented the opening 449 may be divided into a plurality of openings each forming an air outlet and/or one or more additional air outlets may be formed in the upper wall 432.

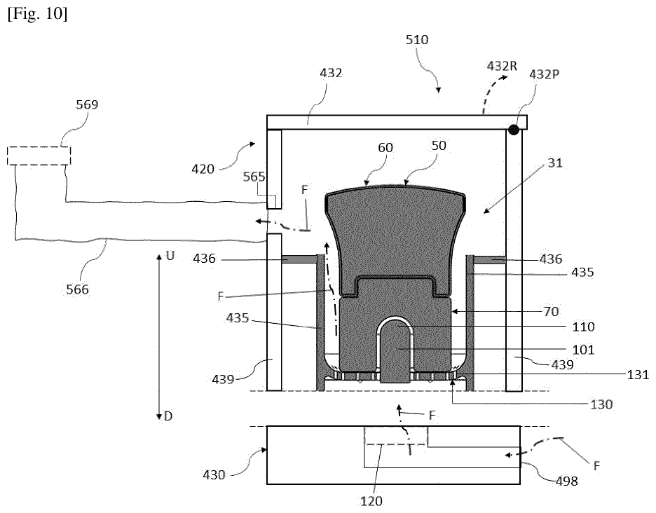

A fifth embodiment of an apparatus 510 is represented schematically in section in FIG. 10. In that figure elements identical to those described above bear identical references and are not described in detail again.

The apparatus 510 differs from the apparatus 410 in that the air outlet of the aeration system is offset relative to frame 430. To be more specific, a communication opening 565 is made in the peripheral wall 439 and places the receptacle 31 in communication with an outlet channel 566. The outlet channel 566 establishes fluid communication of the receptacle 31 with an air outlet 569 via the communication opening 565.

In operation the fan 120 directs a flow of air from the air intake 498 to the evaporation surfaces consisting of the external walls of the wick and then to the communication opening 565 and then to the air outlet 569 via the outlet channel 566. The chain-dotted line arrows F in FIG. 10 indicate the sense and the direction of this flow of air. Thus when the fan 120 and the electrical resistance 110 are functioning the apparatus 510 generates a flow of air charged with evaporated substance exiting via the air outlet 569. In other words the air outlet 569 here forms the single air outlet of the aeration system of the apparatus 510, which air outlet may be at a certain distance from the removable assembly 50. Thus the apparatus 510 can enable diffusion of a flow of air charged with evaporated substance at a certain distance from the removable assembly 50.

The air outlet 569 can take very many forms, for example the form of a nozzle, a meshed orifice, etc. The air outlet 569 may have any orientation and/or be protected by a protective element that is not represented provided that the latter enables the flow of air to exit via the outlet channel 566 as described.

In accordance with variants that are not represented a plurality of outlet channels 566 may be provided, each of these outlet channels 566 being associated with a communication opening 565 at an air outlet 569, and/or one or more additional air outlets may be formed in the upper wall 432.

It may be envisaged that the internal separator wall 336 and/or the various connections of the wick 70 to the storage container 60 described hereinabove are employed in the apparatus 410 or in the apparatus 510.

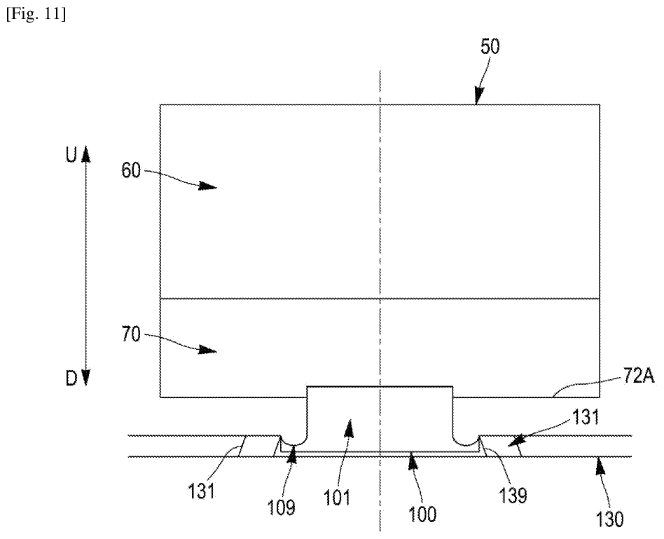

FIG. 11 is a schematic view in section of a variant of the air guide part 130, which variant may be used in all the variants of the apparatus described to this point. Elements identical to those described above bear identical references and are not described in detail again. The elements represented in FIG. 11 are represented only schematically for explanatory purposes.

In this variant the heating member 100 includes, for example at the level of its rod 101, a depression 109 under the lower surface 72A of the wick 70 in the downward direction D. The depression 109 has a concavity facing toward the lower surface 72A of the wick 70.

Consequently dripping of the substance in the liquid state occurs at the level of the wick 70 because of the heating of the wick 70 by the heating member 100, the substance that has dripped in this way accumulating in the depression 109. The substance is therefore prevented from dripping toward other elements of the apparatus, such as the fan 120 for example. On the other hand the substance accumulated in the depression 109 can evaporate because of its relatively high temperature and/or because of the action of the flow of air generated by the fan 120. The substance that has dripped can therefore be evaporated and then evacuated.

The rod 101 and/or the air guide member 130 may be various shapes so as to favor the flow of the substance that has dripped toward the depression 109, for example with a slight downward inclination in the direction of the depression 109. On the other hand the through-holes 131 may be at a minimal distance from the depression 109, for example 3 mm or more, so as to prevent the substance that has dripped passing through the through-holes.

In variants that are not represented the depression 109 is formed in the air guide member 130 and not in the heating member 100 or the air guide member 130 and the heating members 100 each include a depression 109.

Embodiments in which the substance is in the liquid state at ambient temperature have been described to this point. However, the substance is alternatively in the solid state at ambient temperature. For example the substance may have at atmospheric pressure a melting point above 30° C., for example between 30° C. and 40° C. inclusive. In this case the storage container 60 contains the substance in the solid state at ambient temperature. Heating of the wick 70 by the heating member 100 causes local melting of the substance in the vicinity of the drain orifice 61. The substance that has therefore become a liquid can then flow either through the wick 70 or as described above.

Although the invention has been described in connection with a plurality of particular embodiments it is obvious that it is in no way limited to them and that it encompasses all technical equivalents and combinations of the means described if the latter fall within the scope of the invention.

The use of the verb “to include” or to “comprise” and conjugate forms thereof does not exclude the presence of elements or steps other than those stated in a claim.

In the claims any reference sign between parentheses should not be interpreted as a limitation of the claim.

1. A diffusing apparatus (10; 210; 310; 410, 510) for dispersing into the air in the vapor state a substance in the liquid or solid state at ambient temperature, the diffusing apparatus including:
a receptacle (31);
a storage container (60) for containing the substance and including a drain orifice (61), the storage container (60) being received in the receptacle (31) so that the drain orifice (61) is oriented in a downward direction (D) of the diffusing apparatus;
a dispensing member positioned at the outlet of the drain orifice (61) and connected to the drain orifice (61), the dispensing member comprising a porous body (70) having an evaporation surface situated in the receptacle (31) and being situated beyond the drain orifice (61) in the downward direction (D) of the diffusing apparatus;
a heating member (100) arranged on or in the porous body (70) so as to control a flow of the substance through the porous body (70); and
an aeration system comprising an air intake (98), at least one fan (120) and at least one air outlet (33; 449; 569), the aeration system being configured to create a flow of air from the air intake (98) to said at least one air outlet and sweeping the evaporation surface in an upward direction (U) of the diffusing apparatus (10; 210; 310; 410, 510) opposite the downward direction (D),
the diffusing apparatus arranged so that the downward direction (D) is toward the bottom and the upward direction (U) is toward the top relative to the acceleration due to gravity; and
wherein the substance is a liquid substance that has a viscosity variable as a function of temperature, said viscosity being such that in the position of use the substance cannot flow through the porous body (70) at any ambient temperature below a first temperature, the first temperature being above 0° C., and the substance flows through the porous body (70) at a second temperature above the first temperature.

2. The diffusing apparatus (10; 210; 310) as claimed in claim 1 in which the receptacle (31) discharges into the open air via an opening (33) oriented in the upward direction (U) of the diffusing apparatus, the opening (33) forming the air outlet or one of said air outlets of the aeration system.

3. The diffusing apparatus (10; 210; 310) as claimed in claim 2 in which the drain orifice (61) is at a first end of the storage container (60) and the storage container (60) has at a second end opposite the first end a divergent portion (62, 63) facilitating holding the storage container and beyond the opening (33) in the upward direction (U) of the diffusing apparatus so as to divert the flow of air exiting the opening (33) to a periphery of the diffusing apparatus.

4. The diffusing apparatus (10; 210; 310; 410; 510) as claimed in claim 1 in which the fan (120) is beyond the porous body (70) in the downward direction (D) of the diffusing apparatus.

5. The diffusing apparatus (10; 210; 310; 410; 510) as claimed in claim 1 in which the air intake (98) is beyond the porous body (70) in the downward direction (D) of the diffusing apparatus.

6. The diffusing apparatus (10; 210; 310; 410; 510) as claimed in claim 1 in which the aeration system further includes an air guide member (130), the air guide member (130) including at least one through-hole (131) facing the evaporation surface.

7. The diffusing apparatus (10; 210; 310; 410; 510) as claimed in claim 1 in which the heating member (100) comprises an electrical resistance (110) placed directly on an exterior surface of the porous body (70) or received in part in an opening (75), preferably a blind opening, in the porous body (70).

8. The diffusing apparatus (10; 210; 310; 410; 510) as claimed in claim 1, wherein the aeration system further includes an air guide member (130), the air guide member (130) including at least one through-hole (131) facing the evaporation surface, wherein the heating member (100) comprises an electrical resistance (110) placed directly on an exterior surface of the porous body (70) or received in part in an opening (75) in the porous body (70), and wherein the heating member (100) extends through the air guide member (130).

9. The diffusing apparatus (10; 210; 310; 410; 510) as claimed in claim 7 further comprising an electronic circuit card (150), the electrical resistance (110) being supplied with electricity by the electronic circuit card (150).

10. The diffusing apparatus (10; 210; 310; 410; 510) as claimed in claim 1, further comprising a control device configured to control the heating member as a function of a setpoint temperature in the porous body (70).

11. The diffusing apparatus (10; 210; 310; 410; 510) as claimed in claim 7, further comprising:
an electronic circuit card (150), the electrical resistance (110) being supplied with electricity by the electronic circuit card (150);
a control device configured to control the heating member as a function of a setpoint temperature in the porous body (70); and
wherein the control device is on the electronic circuit card (150).

12. The diffusing apparatus (10; 210; 310; 410; 510) as claimed in claim 10 in which the control device is further configured to control an operating speed of the fan (120).

13. (canceled)

14. The diffusing apparatus (10; 210; 310; 410; 510) as claimed in claim 1 in which the substance includes at least one compound selected from the group formed by odoriferous agents usable on man or on animals, semiochemical substances, cosmetic agents, essential oils, perfumes, disinfectants, odor neutralizing agents and phytosanitary and agricultural agents.

15. The diffusing apparatus (10; 210; 310; 410; 510) as claimed in claim 14 in which the substance includes at least one compound selected from the group formed by odoriferous agents usable on man, cosmetic agents, essential oils, perfumes, disinfectants, odor neutralizing agents.

16. A method of using the of a diffusing apparatus (10; 210; 310; 410; 510) of claim 1, comprising:
for dispersing into the air in the vapor state a substance in the liquid or solid state at ambient temperature in which the diffusing apparatus is in the position of use, the downward direction (D) of the diffusing apparatus being toward the bottom relative to the acceleration due to gravity.

17. The method of claim 16 in which the diffusing apparatus (10; 210; 310; 410; 510) is in a closed space or in a place sheltered from precipitation.
